(19) United States
(12) Patent Application Publication
(10) Pub. No.: US 2013/0225873 A1
(43) Pub. Date: Aug. 29, 2013

(54) Promoted Ruthenium Catalyzed Conversion of Syngas to Alcohols

(71) Applicants: Jan Hendrik Blank, Doha (QA); David John Cole-Hamilton, St. Andrews (GB); Robert Thomas Hembre, Johnson City, TN (US); James Allen Ponasik, JR., Kingsport, TN (US)

(72) Inventors: Jan Hendrik Blank, Doha (QA); David John Cole-Hamilton, St. Andrews (GB); Robert Thomas Hembre, Johnson City, TN (US); James Allen Ponasik, JR., Kingsport, TN (US)

(73) Assignee: EASTMAN CHEMICAL COMPANY, Kingsport, TN (US)

(21) Appl. No.: 13/594,537

(22) Filed: Aug. 24, 2012

Related U.S. Application Data
(60) Provisional application No. 61/527,329, filed on Aug. 25, 2011.

(51) Int. Cl. C07C 29/157; B01J 31/28

(52) U.S. Cl. CPC C07C 29/157; B01J 31/28
USPC 568/840; 502/164; 502/161

(57) ABSTRACT

This invention concerns a promoted catalyst system for making one or more alkanols from synthesis gas. The catalyst system contains a ruthenium compound and a halogen promoter dispersed in a low-melting tetraorganophosphonium salt. The halogen promoter is a compound capable of generating HX (where X═Cl, Br, or I) under reaction conditions. The invention also concerns a process for selectively preparing one or more alkanols from synthesis gas using the promoted catalyst system.

### CROSS-REFERENCE TO RELATED APPLICATION

In accordance with 35 U.S.C. §119(e), this application claims the benefit of the filing date of U.S. Provisional Patent Application No. 61/527,329, filed on Aug. 25, 2011; the entire content of which is hereby incorporated by reference.

### FIELD OF THE INVENTION

The invention generally relates to the field of catalysis and, in particular, to promoted-ruthenium catalyzed conversion of synthesis gas to alcohols.

### BACKGROUND OF THE INVENTION

Processes capable of directly converting synthesis gas (a mixture of carbon monoxide and hydrogen) to C₂-oxygenates such as ethanol, acetic acid, or ethylene glycol have long been sought to improve the cost and efficiency of chemicals production from syngas. Syngas may be derived from a variety of feedstocks such as natural gas, coal, petcoke, and biomass; thus, technology employing it as a carbon source may be applied in a range of economic scenarios. To date, only methanol and Fischer-Tropsch hydrocarbons are produced directly from syngas on a billion-pound scale. Technology acceptable for large-scale production of C₂ commodities requires improvements in rate and volume productivity.

The value of ruthenium as a homogeneous catalyst for the conversion of syngas to a mixture of normal alcohols was first reported by DuPont in 1950 (Gresham, U.S. Pat. No. 2,535,060 and Howk & Hager, U.S. Pat. No. 2,549,470). The beneficial influence of halides on homogeneous ruthenium catalysts was reported by Exxon in 1979 (Bradley, U.S. Pat. No. 4,421,862). A series of patents from Texaco, primarily of John Knifton, from 1980-87 established the use of phosphonium salts, particularly tetrabutylphosphonium bromide, in combination with ruthenium-containing catalysts as useful for production of ethylene glycol, ethanol, acetaldehyde, and acetic acid. Thus, ruthenium catalysts in a medium composed of a phosphonium bromide melt have long been known to catalyze the hydrogenation of carbon monoxide to yield methanol, ethanol, acetic acid, and ethylene glycol, along with small amounts of related byproducts.

Yet, there is still a need in the art to develop more efficient catalysis for the conversion of synthesis gas to C₁₊-oxygenates. There is also a need to provide a direct route to useful C₂-derivatives, which eliminates the need for the intermediate manufacture of methanol, in order to improve the economic viability of gas-to-liquid (GTL) processes. The present invention aims to address these needs as well as others, which will become apparent from the following description and claims.

### SUMMARY OF THE INVENTION

In one aspect, the present invention provides a catalyst system for making one or more alkanols from a mixture of carbon monoxide and hydrogen. The catalyst system comprises a ruthenium compound and a halogen compound dispersed in a low-melting tetraorganophosphonium salt. The halogen compound is capable of generating HX under reaction conditions and is present in an amount effective to increase production of the one or more alkanols compared to a catalyst system without the halogen compound, where X is Cl, Br, or I.

In a second aspect, the present invention provides a process for preparing one or more alkanols. The process comprises the step of contacting a mixture of carbon monoxide and hydrogen with a catalyst system comprising a ruthenium compound and a halogen compound dispersed in a low-melting tetraorganophosphonium salt under conditions effective to produce one or more alkanols. The halogen compound is capable of generating HX under reaction conditions and is present in an amount effective to increase production of the one or more alkanols compared to a catalyst system without the halogen compound, where X is Cl, Br, or I.

### BRIEF DESCRIPTION OF THE DRAWINGS

FIG. 1 is a graph of the amount of certain products produced (methanol, ethanol, and ethylene glycol) from Examples 4-12 as a function of the molar ratio of [HPBu₃]Br/Ru.

FIG. 2 is the ³¹P NMR spectrum of [HPBu₃]Br (δ=11.66 ppm, dm, ¹J_{PH}=485 Hz) from Example 1.

FIG. 3 is the ¹H undecoupled ³¹P NMR spectrum of [PBu₄]Br (δ=33.52) from Example 2.

FIG. 4 is the ¹H decoupled ³¹P NMR spectrum of [PBu₄]Br (δ=33.52) from Example 2.

FIG. 5 is the ¹H decoupled ³¹P NMR spectrum of the product solution from Example 1.

FIG. 6 is a graph of the amount of methanol, ethanol, and ethylene glycol produced versus the bromide/ruthenium molar ratio resulting from the bromide/trifluoromethane-sulfonate composition of the tetrabutylphosphonium salt in a series (Examples 17-24) of CO/H₂ conversions.

FIG. 7 is a graph of the amount of methanol, ethanol, and ethylene glycol produced from Examples 25-28 showing the relative influence of tributylphosphonium bromide and hydrogen bromide in promoting and tributylphosphine in suppressing alkanol production.

FIG. 8 is a graph of the amount of certain products produced (methanol, ethanol, 1-propanol, and ethylene glycol) from Examples 29-38 as a function of the molar ratio of HCl/Ru.

FIG. 9 is a graph of the amount of certain products produced (methanol, ethanol, 1-propanol, and ethylene glycol) from Examples 39-46 as a function of the molar ratio of HBr/Ru.

FIG. 10 is a graph of the amount of certain products produced (methanol, ethanol, 1-propanol, and ethylene glycol) from Examples 47-51 as a function of the molar ratio of HI/Ru.

### DETAILED DESCRIPTION OF THE INVENTION

It has been surprisingly discovered that adding a halogen compound capable of generating HX (where X═Cl, Br, or I) under reaction conditions to a reaction medium containing a ruthenium-based catalyst and a low-melting phosphonium halide solvent promotes the system for the production of methanol and/or ethanol. In particular, the halogen compound can increase the rate of alcohol production by 50-100% or more.

The catalyst system according to the invention comprises a ruthenium compound and a halogen compound dispersed in a low-melting tetraorganophosphonium salt.

The ruthenium catalyst component may be chosen from a wide variety of organometallic or inorganic compounds, complexes, etc. For instance, the ruthenium component may be added to the reaction mixture in an oxide form, as in the case of, for example, ruthenium(IV) oxide hydrate, anhydrous ruthenium(IV) dioxide and ruthenium(VIII) tetraoxide. Alternatively, it may be added as the salt of a mineral acid, as in the case of ruthenium(III) chloride hydrate, ruthenium(III) bromide, ruthenium(III) triiodide, tricarbonyl ruthenium(II)iodide, anhydrous ruthenium(III) chloride, and ruthenium nitrate; or as the salt of a suitable organic carboxylic acid, for example, ruthenium(III) acetate, ruthenium propionate, and ruthenium(III) acetylacetonate. The ruthenium component may also be added to the reaction zone as a carbonyl or hydrocarbonyl derivative. Suitable examples include triruthenium dodecacarbonyl; hydrocarbonyls such as H₂Ru₄(CO)₁₃, H₄Ru₄(CO)₁₂, and salts of [HRu₃(CO)₁₁]⁻; and substituted carbonyl species such as the tricarbonylruthenium(II) chloride dimer, [Ru(CO)₃Cl₂]₂, and salts of [Ru(CO₃)X₃]⁻ where X═Cl, Br, or I. Any other ruthenium compound that can generate a soluble ruthenium carbonyl halide complex under the reaction conditions can also be used. The ruthenium compounds can be used individually or as mixtures of two or more ruthenium compounds.

Preferred ruthenium compounds include oxides of ruthenium, ruthenium salts of a mineral acid, ruthenium salts of an organic carboxylic acid, and ruthenium carbonyl or hydrocarbonyl derivatives. Among these, particularly preferred are ruthenium(IV) dioxide hydrate, ruthenium(VIII) tetraoxide, anhydrous ruthenium(IV) oxide, ruthenium acetate, ruthenium(III) acetylacetonate, triruthenium dodecacarbonyl, bis[ruthenium(tricarbonyl)dichloride], bis[ruthenium(tricarbonyl)dibromide], and bis[ruthenium(tricarbonyl)diiodide].

The concentration of ruthenium compound charged to the reaction can be from 0.01 to 30 weight percent of the total weight of the reaction mixture, based on contained ruthenium. The concentration is preferably from 0.2 to 10 weight percent, and most preferably from 0.5 to 5 weight percent.

The halogen catalyst component according to the invention is a compound capable of generating HX (where X═Cl, Br, or I) under reaction conditions. Any source of chlorine, bromine, or iodine capable of generating HX in situ can be used. Examples of such a source include elemental chlorine, bromine, and iodine. Also among the useful compounds are the alkyl halides having, for example, from 1 to 10 carbon atoms, as well as any other organic halide compound capable of producing HX in situ. Illustrative of suitable halide promoters include methyl iodide, butyl iodide, acetyl iodide, hydrogen iodide, cobalt iodide, as well as the corresponding chloride and bromide compounds. Further, mixtures of the elemental halogens and/or the halogen compounds can be used.

A preferred class of halogen promoters capable of generating HX under reaction conditions includes a triorganophosphonium salt having the general formula (II)

[R₁R₂R₃PH][X²] (II)

wherein

R₁, R₂, and R₃ are each independently selected from C₁-C₂₄ alkyl or aryl hydrocarbon groups or functionalized alkyl or aryl groups containing ether, alcohol, ketone, carboxylic acid or ester, amine, amide, thioether, phosphine oxide, nitrile, heteroaromatic, or fluorocarbon groups; and

X² is chloride, bromide, or iodide.

Illustrative examples of suitable triorganophosphonium salts include tributylphosphonium chloride, triphenylphosphonium chloride, tributylphosphonium bromide, triphenylphosphonium bromide, tributylphosphonium iodide, and triphenylphosphonium iodide.

The preferred salts are generally the trialkylphosphonium salts containing alkyl groups having 1-6 carbon atoms, such as methyl, ethyl, and butyl. Tributylphosphonium salts, such as tributylphosphonium bromide, are most preferred for the practice of this invention.

The halogen promoter is charged to the reaction in an amount sufficient to increase production of one or more of the alkanols compared to a catalyst system without the halogen compound. Typically, the halogen promoter is used in an amount sufficient to produce an HX/Ru atom molar ratio of 0.05:1 to 3.5:1 during the reaction. In the case of X═Cl, the ratio may be from 0.05:1 to 3:1, and preferably from 0.1:1 to 2.5:1. In the case of X═Br or I, the ratio may be from 0.05:1 to 1:1, preferably from 0.1:1 to 0.9:1, and more preferably from 0.2:1 to 0.8:1. When a triorganophosphonium salt is used, a triorganophosphonium salt to Ru atom molar ratio of 0.2:1 to 0.6:1 is preferred, particularly when the salt is bromide or iodide based.

According to the invention, the catalyst components are dispersed in a low-melting tetraorganophosphonium salt. By “low melting,” it is meant that the salt melts at a temperature less than the reaction temperature for making the one or more alkanols. Usually, the salt has a melting point of 180° C. or less, and preferably of 150° C. or less.

The low-melting tetraorganophosphonium salt can have the general formula (I):

[R₁R₂R₃R₄P][X¹] (I)

wherein

R₁, R₂, R₃, and R₄ are each independently selected from C₁-C₂₄ alkyl or aryl hydrocarbon groups or functionalized alkyl or aryl groups containing ether, alcohol, ketone, carboxylic acid or ester, amine, amide, thioether, phosphine oxide, nitrile, heteroaromatic, or fluorocarbon groups; and

X¹ is chloride, bromide, or iodide.

Illustrative examples of suitable tetraorganophosphonium salts include tetrabutylphosphonium chloride, heptyltriphenylphosphonium chloride, tetrabutylphosphonium bromide, heptyltriphenylphosphonium bromide, tetrabutylphosphonium iodide, and heptyltriphenylphosphonium iodide.

The preferred salts are generally the tetraalkylphosphonium salts containing alkyl groups having 1-6 carbon atoms, such as methyl, ethyl, and butyl. Tetrabutylphosphonium salts, such as tetrabutylphosphonium bromide, are most preferred for the practice of this invention.

The catalyst system according to the invention is particularly suitable for use in a process for preparing one or more alkanols from syngas. The process comprises contacting a mixture of carbon monoxide and hydrogen with the catalyst system according to the invention under conditions effective to produce one or more alkanols.

The reaction conditions effective to produce one or more alkanols include a temperature from 100° C. to 350° C. Preferably, the temperature is at least 150° C., such as from 150° C. to 280° C. More preferably, the temperature is at least 180° C., such as from 180° C. to 250° C.

The effective reaction conditions also include a CO/H₂ pressure from 500 psi (3.5 MPa) to 20,000 psi (130 MPa) or more. Preferably, the CO/H₂ pressure is at least 1,000 psi (7 MPa), such as from 1,000 psi (7 MPa) to 12,500 psi (86 MPa). More preferably, the CO/H₂ pressure is at least 2,000 psi (14 MPa), such as from 2,000 psi (14 MPa) to 8,000 psi (55 MPa).

The volumetric ratio of carbon monoxide to hydrogen (CO:H₂) in the synthesis gas feed mixture can range from 0.1:1 to 10:1, preferably from 0.25:1 to 4:1, and more preferably from 0.33:1 to 2:1.

The process of this invention can be conducted in a batch, semi-continuous, or continuous fashion. The catalyst system may be initially introduced into the reaction zone batchwise, or it may be continuously or intermittently introduced into such a zone during the course of the synthesis reaction. Operating conditions can be adjusted to optimize the formation of the desired alkanol product. Preferred products include methanol or ethanol or both. The alkanol product may be recovered by methods well known in the art, such as distillation, fractionation, extraction, and the like. A fraction rich in the ruthenium catalyst component may then be recycled to the reaction zone, if desired, and additional products generated.

The mixture of carbon monoxide and hydrogen is typically introduced into the reactor as a gas, while the catalyst components are typically first introduced into the reactor as solids and/or liquids. Under reaction conditions, the tetraorganophosphonium salt should be in the liquid phase as a melt, and the ruthenium and halogen compounds are dispersed in that melt.

As used herein, the terms “synthesis gas” or “syngas” refer to a gas mixture of carbon monoxide (CO) and hydrogen (H₂). The gas mixture used herein did not include carbon dioxide (CO₂).

As used herein, the indefinite articles “a” and “an” mean one or more, unless the context clearly suggests otherwise. Similarly, the singular form of nouns includes their plural form, and vice versa, unless the context clearly suggests otherwise.

While attempts have been made to be precise, the numerical values and ranges described herein should be considered to be approximations. These values and ranges may vary from their stated numbers depending upon the desired properties sought to be obtained by the present invention as well as the variations resulting from the standard deviation found in the measuring techniques. Moreover, the ranges described herein are intended and specifically contemplated to include all sub-ranges and values within the stated ranges. For example, a range of 50 to 100 is intended to include all values within the range including sub-ranges such as 60 to 90 and 70 to 80.

This invention can be further illustrated by the following examples of preferred embodiments thereof, although it will be understood that these examples are included merely for purposes of illustration and are not intended to limit the scope of the invention. Unless otherwise indicated, all percentages are by weight.

### EXAMPLES

Unless stated otherwise, all chemicals were obtained from Sigma Aldrich and used as received. Air-sensitive compounds were handled under N₂ using standard Schlenk techniques. NMR spectra where recorded on a Varian 300 NMR or Bruker AM 300/400 NMR spectrometers. ¹H chemical shifts were referenced to the solvent: CD₂HCN δ 1.95 (p, J=2.5 Hz). ³¹P NMR spectra were referenced to external H₃PO₄. Gas chromatography analysis was performed using a Supelcowax-10 Capillary Column (60 m×0.32 mm×1.0 μm film thickness) using an Agilent 6890N Network GC system equipped with a flame ionisation detector. For identification, a HP 6890 series GC system equipped with a HP 5973 mass selective detector was used. Both machines used 1 mL min⁻¹ He carrier gas flow and 250° C. injector and detector temperatures. The temperature was programmed as follows: 50° C., hold 3 minutes, ramp 20° C. min⁻¹, 150° C., hold 5 min, ramp 20° C. min-1, 220° C., hold 13 minutes. The split ratio was 1:100.

### Example 1

### Preparation of Tributylphosphonium Bromide [HPBu₃]Br

Dry HBr was generated in situ and transferred continuously into a flask containing PBu₃ dissolved in chilled diethylether. The total setup consisted of two flasks connected through a glass transfer tube, fitted with a plug of P₂O₅. In one flask fitted with a stir bar, H₃PO₄ (85%, 33 mL) was degassed using three freeze-pump-thaw cycles, and subsequently dried using an excess of P₂O₅ (30 g). Then dry KBr (65 g) was added and the system was degassed once more before being fitted with the glass connection tube. Next, a three-neck flask was evacuated, dried, fitted with a stir bar and added to the other end of the glass connection tube under N₂. Next, diethyl ether (250 mL) was added followed by PBu₃ (32 mL). The flask containing the PBu₃ was subsequently chilled using a dry ice, acetone bath. The flask containing the KBr salt was then heated until HBr was liberated. Immediately, formation of the product was visible as a white precipitate in the flask containing the PBu₃. The reaction was controlled by control of the heating of the flask containing the KBr. This procedure was continued until no more HBr was liberated. The white product was then collected by filtration and washed 3 times using dry diethylether (50-60 mL). After in vacuo evaporation, this yielded a fine, white powder (31.2 g, 90%).

¹H NMR (CD₃CN, δ (ppm)): 6.50 (1H, d, ¹J_{PH}=485.2 Hz, Bu₃P—H), 2.29 (6H, m, P—(CH₂—C₃H₇)₃), 1.62 (6H, m, [HP—(CH₂—CH₂—C₂H₅)₃]⁺), 1.48 (6H, m, [HP—(C₂H₄—CH₂—CH₃)]⁺), 0.96 (9H, t, ³J_{HH}=7.2 Hz, [HP—(C₃H₆—CH₃)₃]⁺).

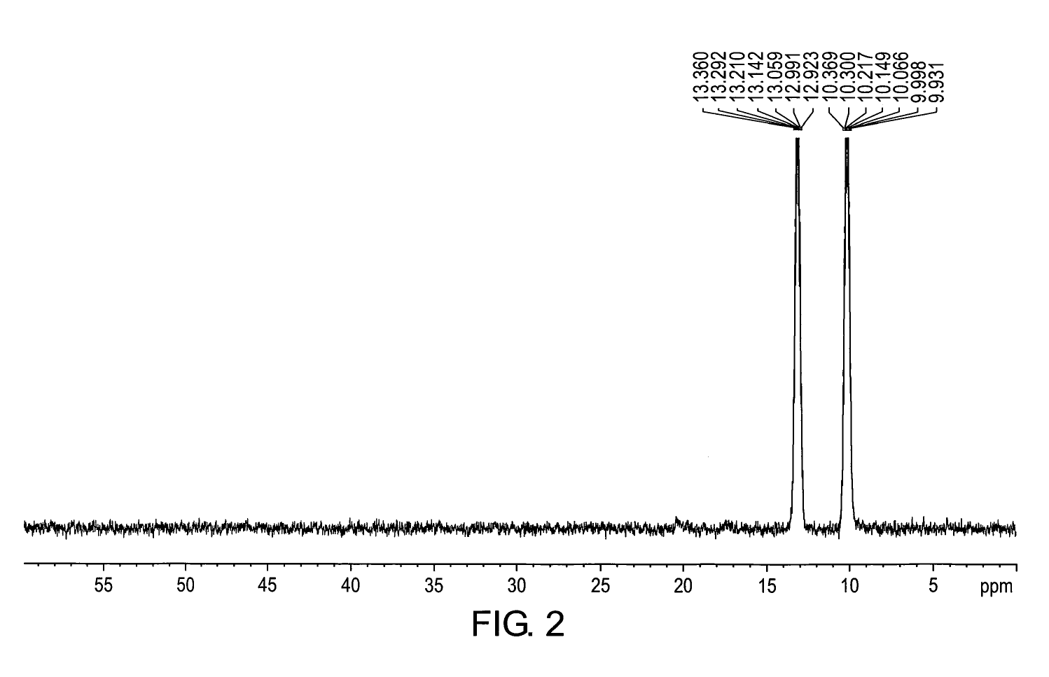

³¹P NMR (CD₃CN, δ (ppm)): 11.66 (δ, ¹J_{PH}=484.9 Hz). The spectrum is shown in FIG. 2.

### Example 2

### Purification of Tetrabutylphosphonium Bromide [PBu₄]Br

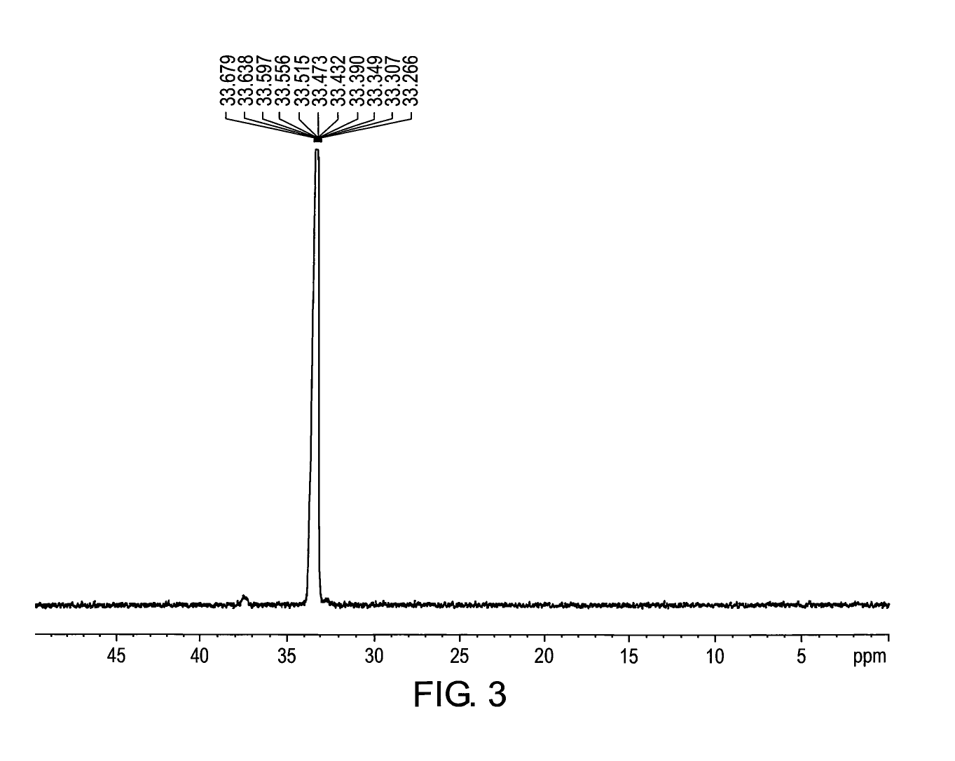

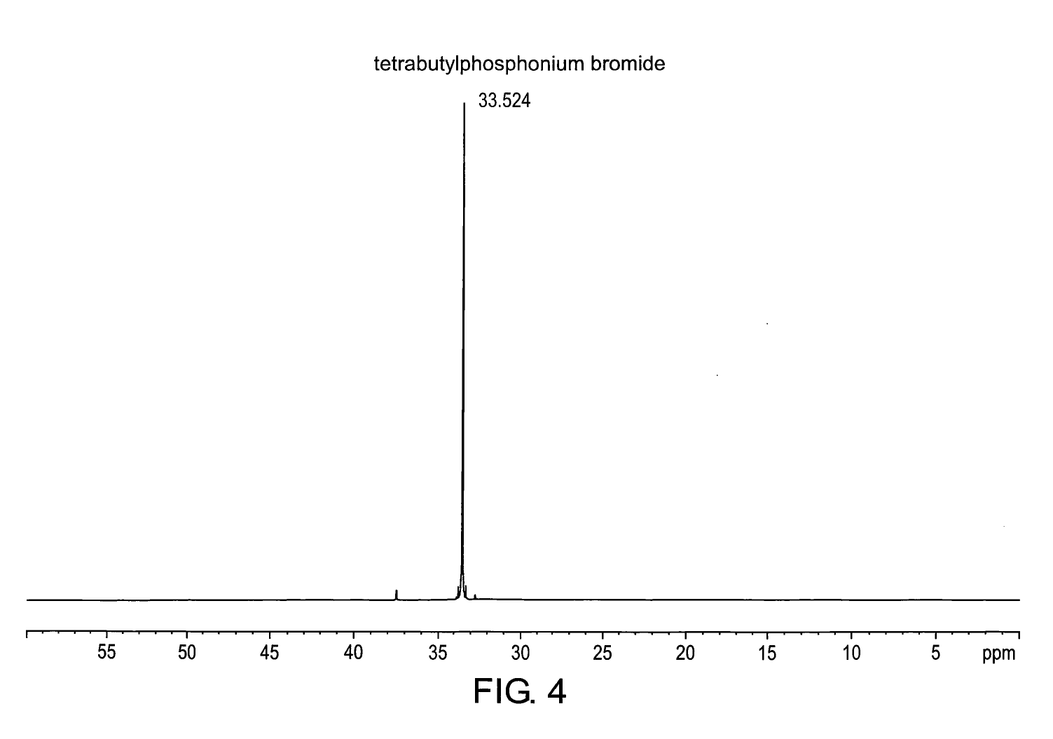

The typical procedure for purification of [PBu₄]Br was to dissolve it in acetone and then precipitate it by addition of diethylether. For example, [PBu₄]Br (200 g) was dissolved in acetone (125 mL). To this diethyl ether (600 mL) was added slowly. The [PBu₄]Br precipitated and was collected on a glass frit, washed with diethyl ether (3×200 mL), and dried in vacuo yielding 192.7 g of product (96%). The δ 11.7 doublet of [HPBu₃]Br was undetectable in the ¹H coupled ³¹P spectrum (FIG. 3), but the decoupled spectrum revealed a trace (FIG. 4). The small peak at δ 37 is attributed to (tri-n-butyl))(s-butyl)phosphonium salt.

### Example 3

### Preparation of Tetrabutylphosphonium Chloride [PBu₄]Cl

To a flask fitted with a stirrer and a reflux condenser containing dry and degassed diethylether (100 mL), 190.9 mL of P(C₄H₇)₃ (154.7 g, 0.764 mol) was added. 96.5 mL of butyl chloride (84.938 g, 0.918 mol) was added and the solution was allowed to stir under reflux for 2 weeks. This yielded very small amounts of crystalline white product. To speed up the reaction, the diethylether was stripped off by distillation and the reaction was continued under reflux for 1 and a half days, while the product layer continuously grew in size. The mixture was cooled to room temperature leading to the formation of a solid product layer, and this was topped by 50 mL of diethylether (for storage). The diethylether was later decanted and the product was then dissolved in 100 mL of acetone. After addition of 1200 mL of diethylether, the product precipitated. The precipitate was filtered, washed with 3×150 mL of diethylether, and dried in vacuo to yield white powder (161.8 g, 0.680 mol, 89%). ¹H NMR (CD₃CN, δ (ppm)): 2.22 (8H, P—(CH₂—C₃H₇)₄); 1.49 (16H, m, [P—(CH₂—CH₂—CH₂—CH₃)₄]⁺); 0.95 (12H, [P—(C₃H₆—CH₃)₄]⁺). ³¹P NMR (CD₃CN, δ (ppm)): 33.55 ([PBu₄]Cl) 37.51 (impurity: [P(s-C₄H₉)(n-C₄H₉)₃]Cl).

### Example 4

### Preparation of Tetrabutylphosphonium Iodide [P(C₄H₉)₄]I

To a flask fitted with a stirrer and reflux condenser containing dry and degassed diethylether (100 mL), PBu₃ (135 mL, 110.7 g, 0.547 mol) and subsequently butyl iodide (75 mL, 120.8 g, 0.657 mol) was added, and the solution was allowed to stir for 48 hours. This yielded a white precipitate, which was filtered, and then washed 3 times with 100 mL diethylether. The product was then filtered again and dried in vacuo to yield a white soft solid (154.8 g, 0.401 mol, 73.2%). ¹H NMR (CD₃CN, δ (ppm)): 2.19 (8H, m, [P—(CH₂—C₃H₇)₄]); 1.50 (16H, m, ³J_{HH}=7.0 (C₃); 7.28 (C₂) Hz, [P—(CH₂—CH₂—CH₂—CH₃)₄]⁺); and 0.96 (12H, t, ³J_{HH}=6.9 Hz, [P—(C₃H₆—CH₃)₄]⁺). ³¹P NMR (CD₃CN, δ (ppm)): 33.63 (d, ²J_{PH}=6.5 Hz) 37.63 (impurity: [P(s-C₄H₉)(n-C₄H₉)₃]I). Tetrabutylphosphonium iodide can be stored under N₂ for prolonged times without discoloration.

### Example 5

### Preparation of Tetrabutylphosphonium Trifluoromethanesulfonate [P(C₄H₉)₄]O₃SCF₃

In a flask fitted with a stirrer, CF₃SO₃K (50.233 g, 0.267 mol) was added to [PBu4]Br (82.353 g, 0.243 mol). The mixture was dissolved in 100 mL of water, and a precipitate was formed almost instantly, but the mixture was stirred overnight. The liquid was decanted and the precipitate was washed 3 times using 30 mL of water. After filtration, the mixture was dried in vacuo yielding an off-white solid (97.5 g, 0.239 mol, 98%).

### General Procedure for Synthesis Gas Conversion

The solids, [Bu4P]Br, Ru₃(CO)₁₂, and [Bu₃PH]Br, when used, where weighed out and added to the clean and dry autoclave. Next, the autoclave was screwed onto the holder and the reactor was filled with approximately 145 psi (1 MPa) CO/H₂ (50:50 v/v) and then vented. This purging sequence was repeated at least 6 or 7 times to ensure adequate removal of air. The system was then filled with syngas to approximately 2,465 psi (17 MPa) and tested for leaks. The heater was attached, set to 200° C., and switched on with continuous stirring.

When the reactor had reached the desired temperature of 200° C., the reactor pressure was adjusted to 3,626 psi (25 MPa). During the reaction, the pressure was held continuously at 3,626 psi (25 MPa), by the addition of syngas from an attached ballast vessel. The pressure of the ballast vessel was monitored throughout the reaction by using computerized logging equipment. The reaction was allowed to run for 4 hrs, after which the heater was switched off and decoupled to ensure swift cooling.

When the temperature of the autoclave dropped below 30° C., the excess pressure was vented from the autoclave, and the product mixture was inspected. The usual color was red. The red liquid was poured into a glass flask, weighed, and the product mixture distilled under reduced pressure by increasing the temperature to 250° C. The condensed vapors where collected in a liquid N₂ trap. Next, the products were diluted using a stock solution of 2% acetonitrile (internal standard) in N-methylpyrrolidone (NMP) (v/v) (5 mL total volume). This mixture was analyzed using GC-FID. The retention times and product identities were previously determined using GC-MS.

### Example 6

[PBu₄]Br (14.800 g, 43.6 mmol), [HPBu₃]Br (0.166 g, 0.58 mmol), and Ru₃(CO)₁₂ (0.499 g, 2.34 mmol) were added to the reactor. The reactor was purged and pressurized with syngas (2,465 psi (17 MPa), CO:H₂=1:1). The system was heated to 200° C., and then the pressure was adjusted to 3,626 psi (25 MPa). The reaction was allowed to stir for 4 hours under a constant pressure of 3,626 psi (25 MPa), make-up syngas (1:1) being fed from a ballast vessel before the heating was switched off. Next the reactor was allowed to cool to below 30° C., and the excess gas was vented. The red liquid product was distilled to yield the products. Back calculations of the weight of the product liquid before and after distillation showed that the reactor contained 1.2 g of product. The GC analysis mainly showed formation of methanol, ethanol, propanol, butanol, acetic acid, ethylene glycol, 2-methoxyethanol, 2-ethoxyethanol, and minor amounts of other compounds, most of which were not identified or quantified. The quantities produced are reported in Table 1 below.

TABLE 1

| | Quantity |
|---|---|
| Product | (mmol) |
| Methanol | 25.496 |
| Ethanol | 5.994 |
| 1-Propanol | 0.285 |
| 1-Butanol | 0.017 |
| Acetic Acid | 0.774 |
| Ethylene Glycol | 2.872 |
| 2-Methoxyethanol | 0.321 |
| 2-Ethoxyethanol | 0.104 |

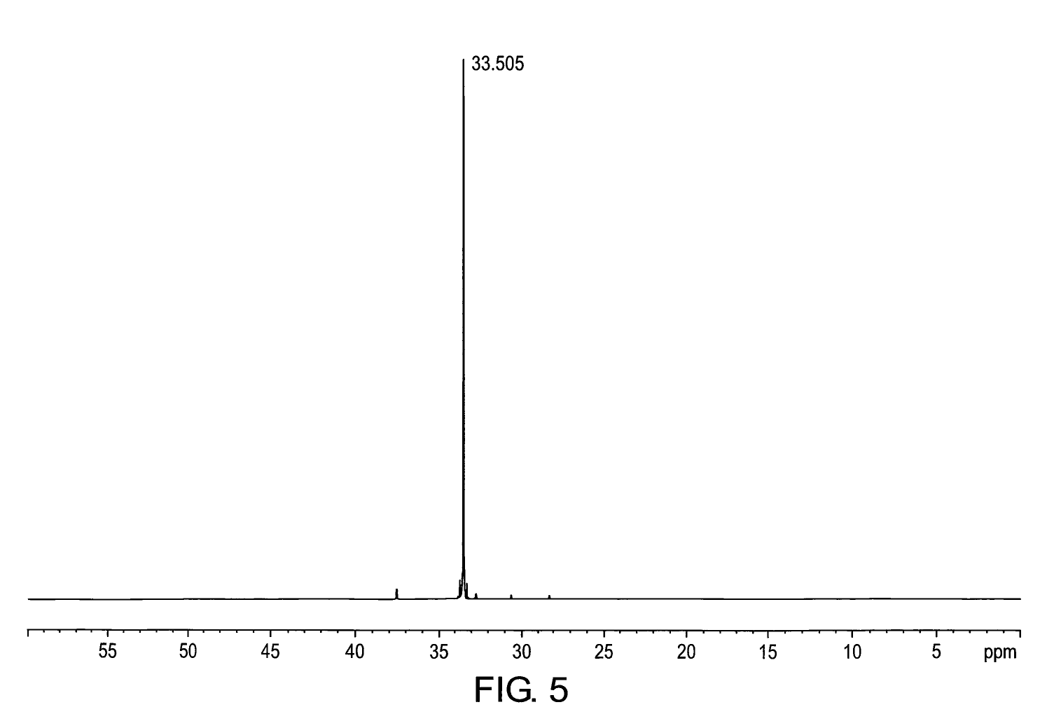

The ³¹P NMR of the product liquid after the reaction did not show the presence of any [HPBu₃]Br. See FIG. 5.

### Examples 7-16

The procedure of Example 6 was followed using the amounts of [PBu₄]Br, [HPBu₃]Br, and Ru₃(CO)₁₂ listed in Table 2 below.

TABLE 2

| Example | [PBu₄]Br | [HPBu₃]Br | Ru₃(CO)₁₂ | Molar Ratio of |
|---|---|---|---|---|
| No. | g (mmol) | mg (mmol) | mg (mmol) | [HPBu₃]Br/Ru |
| 7 | 14.800 (43.6) | 166.5 (0.6) | 499.1 (0.78) | 0.25 |
| 8 | 14.602 (43.0) | 332.9 (1.2) | 500.0 (0.79) | 0.05 |
| 9 | 14.203 (41.9) | 664.2 (2.4) | 499.3 (0.78) | 1.00 |
| 10 | 14.921 (44.0) | 67.4 (0.2) | 499.6 (0.79) | 0.10 |
| 11 | 15.001 (44.2) | — | 500.1 (0.79) | 0.00 |
| 12 | 14.403 (42.5) | 498.8 (1.8) | 499.1 (0.78) | 0.75 |
| 13 | 14.702 (43.3) | 249.3 (0.9) | 500.4 (0.79) | 0.37 |
| 14 | 14.502 (42.7) | 414.5 (1.5) | 499.1 (0.78) | 0.62 |
| 15 | 14.303 (42.2) | 582.2 (2.1) | 500.0 (0.79) | 0.88 |
| 16 | 14.503 (42.7) | 414.9 (1.5) | 499.7 (0.79) | 0.62 |

The product yields, in millimoles, are displayed in Table 3 below. In Table 3, tr=trace and nd=not detected.

Example 16 was an experiment with increased stirring to check the mass transport effects.

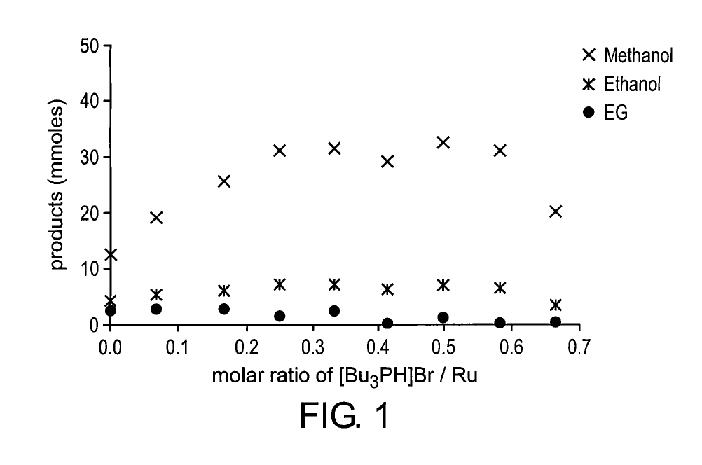

The amount of certain products produced (methanol, ethanol, and ethylene glycol) is shown graphically in FIG. 1 as a function of the molar ratio of [HPBu₃]Br/Ru.

TABLE 3

| Product | Example No. | | | | | | | | |
|---|---|---|---|---|---|---|---|---|---|
| Yield (mmol) | 7 | 8 | 9 | 10 | 11 | 12 | 13 | 14 | 15 |
| Methanol | 31.43 | 20.18 | 19.08 | 12.50 | 32.45 | 31.03 | 29.04 | 31.04 | 34.05 |
| Ethanol | 7.19 | 3.37 | 5.34 | 4.34 | 7.02 | 7.15 | 6.30 | 6.48 | 7.42 |
| 1-Propanol | 0.30 | 0.17 | 0.30 | 0.29 | 0.27 | 0.32 | 0.21 | 0.23 | 0.28 |
| 1-Butanol | tr | 0.02 | 0.02 | 0.02 | 0.02 | 0.02 | 0.01 | 0.02 | 0.02 |
| Acetic Acid | 0.90 | 0.28 | 0.74 | 0.68 | 1.01 | 0.61 | 0.26 | 0.26 | 0.27 |
| Ethylene Glycol | 2.41 | 0.41 | 2.76 | 2.59 | 1.12 | 1.48 | 0.18 | 0.19 | 0.43 |
| 2-Methoxy ethanol | 0.31 | 0.07 | 0.32 | 0.25 | 0.23 | 0.36 | 0.16 | 0.13 | 0.26 |
| 2-Ethoxy ethanol | 0.10 | 0.01 | 0.125 | 0.13 | 0.01 | 0.02 | 0.01 | tr | 0.07 |
| Dimethoxy Methane | 0.94 | 0.09 | 0.69 | nd | 0.64 | 1.39 | 1.01 | 0.66 | 1.08 |

### Examples 17-24

A series of experiments was run to examine the effect of bromide in the phosphonium salt as a promoter on the hydrogenation of carbon monoxide with Ru₃(CO)₁₂ catalyst in molten [Bu₄P]Br. A procedure similar to that described in Example 6 was carried out in each case with a reaction temperature of 195° C., CO/H₂ pressure of 3,626 psi (25 MPa), and CO/H₂ volumetric ratio of 1:1 for 4 h for the compositions detailed in Table 4.

TABLE 4

| | | | | molar |
|---|---|---|---|---|
| Example | [PBu₄]Br | [PBu₄]O₃SCF₃ | Ru₃(CO)₁₂ | ratio of |
| No. | g (mmol) | g (mmol) | mg (mmol) | [PBu₄]Br/Ru |
| 17 | 0.000 (0.0) | 15.001 (36.7) | 499.4 (2.34) | 0.00 |
| 18 | 1.593 (4.7) | 13.408 (32.8) | 499.4 (2.34) | 2.00 |
| 19 | 2.388 (7.0) | 11.612 (28.4) | 500.0 (2.35) | 2.98 |
| 20 | 3.184 (9.4) | 11.815 (28.9) | 499.5 (2.34) | 4.00 |
| 21 | 3.980 (11.7) | 11.020 (27.0) | 499.3 (2.34) | 5.00 |
| 22 | 7.961 (23.5) | 7.038 (17.2) | 500.2 (2.35) | 10.00 |
| 23 | 11.942 (35.2) | 3.058 (7.5) | 500.3 (2.35) | 14.98 |
| 24 | 15.924 (46.9) | 0.000 (0.0) | 500.0 (2.35) | 19.95 |

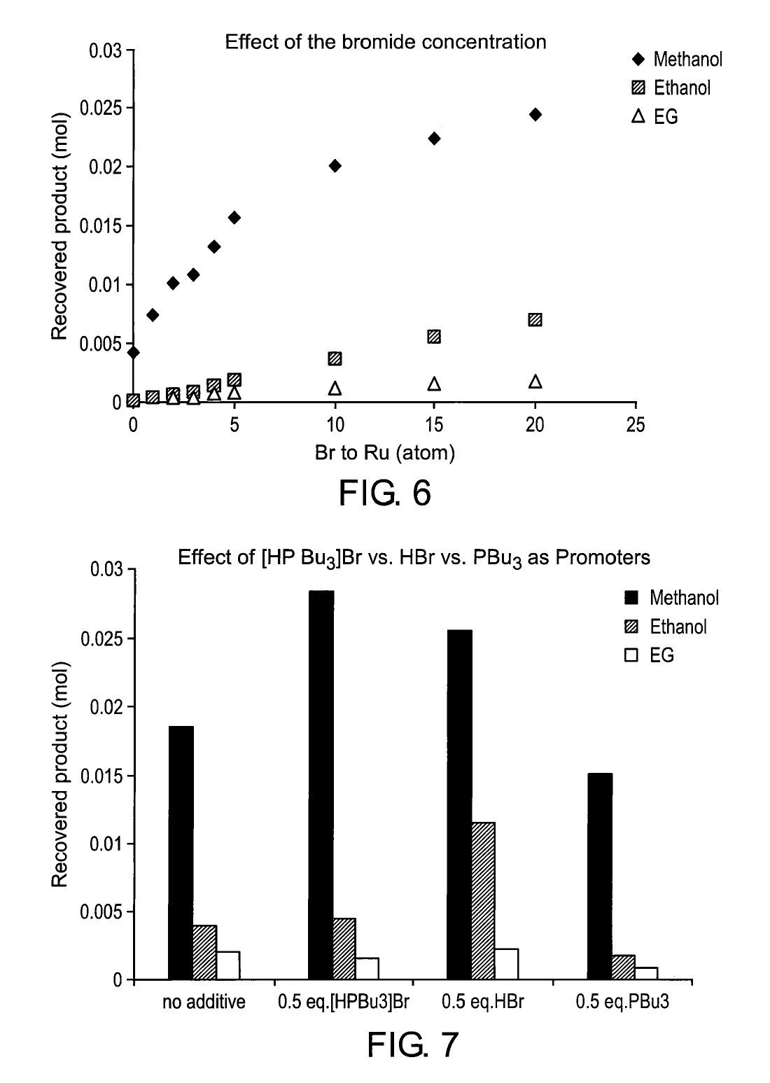

The amount of methanol, ethanol, and ethylene glycol produced is shown graphically in FIG. 6 as a function of the molar ratio of bromide/ruthenium.

### Examples 25-28

A series of four experiments was run to compare the effects of [HPBu₃]Br and HBr as promoters and PBu₃ as an additive on the hydrogenation of carbon monoxide with Ru₃(CO)₁₂ catalyst in molten [Bu₄P]Br. A procedure similar to that described in Example 6 was carried out in each case with a reaction temperature of 200° C., CO/H₂ pressure of 3,626 psi (25 MPa), and CO/H₂ volumetric ratio of 1:1 for 4 h.

The amounts of methanol, ethanol, and ethylene glycol produced in each example is shown graphically in FIG. 7.

### Examples 29-38

A series of ten experiments was run to examine the effect of HCl as a promoter on the hydrogenation of carbon monoxide with Ru₃(CO)₁₂ catalyst in molten [Bu₄P]Cl. A procedure similar to that described in Example 6 was carried out in each case with a reaction temperature of 195° C., CO/H₂ pressure of 3,626 psi (25 MPa), and CO/H₂ volumetric ratio of 1:1 for 4 h for the compositions detailed in Table 5.

TABLE 5

| Example | HCl | Ru₃(CO)₁₂ | PBu₄Cl |
|---|---|---|---|
| No. | mg (mmol) | mg (mmol) | g (mmol) |
| 29 | 1.69 (0.05) | 125.4 (0.20) | 13.021 (44.2) |
| 30 | 3.40 (0.09) | 124.8 (0.20) | 13.006 (44.1) |
| 31 | 5.35 (0.15) | 125.4 (0.20) | 12.991 (44.1) |
| 32 | 10.62 (0.29) | 125.3 (0.20) | 12.949 (43.9) |
| 33 | 16.14 (0.44) | 124.6 (0.20) | 12.906 (43.8) |
| 34 | 21.24 (0.58) | 125.5 (0.20) | 12.862 (43.6) |
| 35 | 32.28 (0.89) | 125.4 (0.20) | 12.775 (43.3) |
| 36 | 42.90 (1.18) | 124.9 (0.20) | 12.689 (43.0) |
| 37 | 64.14 (1.76) | 125.2 (0.20) | 12.516 (42.4) |
| 38 | 74.76 (2.05) | 125.4 (0.20) | 12.430 (42.2) |

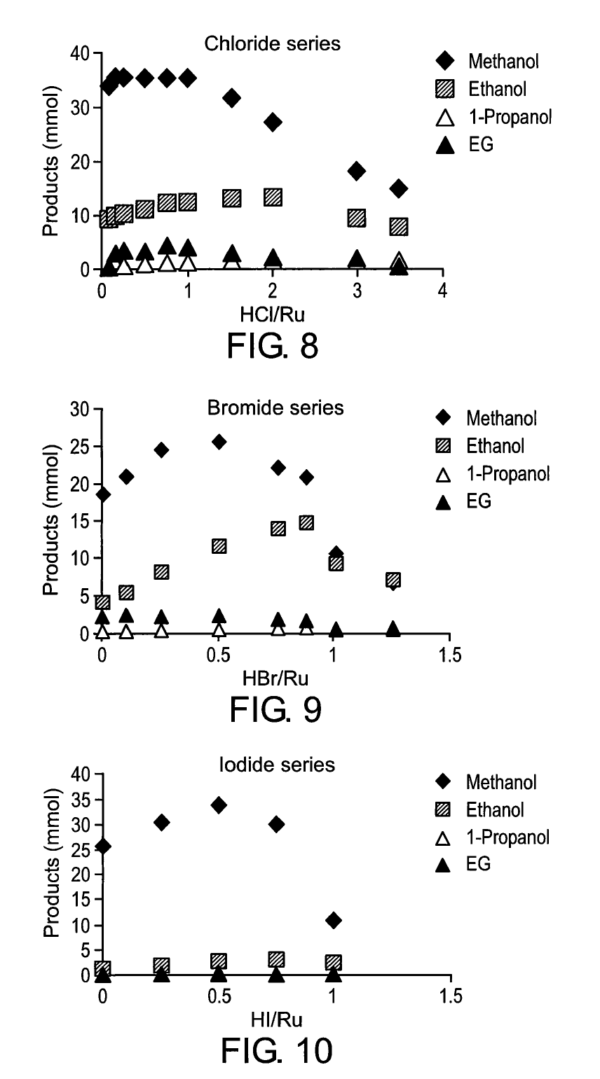

The amount of certain products produced (methanol, ethanol, 1-propanol, and ethylene glycol) is shown graphically in FIG. 8 as a function of the molar ratio of HCl/Ru.

### Examples 39-46

A series of eight experiments was run to examine the effect of HBr as a promoter on the hydrogenation of carbon monoxide with Ru₃(CO)₁₂ catalyst in molten [Bu₄P]Br. A procedure similar to that described in Example 6 was carried out in each case with a reaction temperature of 195° C., CO/H₂ pressure of 3,626 psi (25 MPa), and CO/H₂ volumetric ratio of 1:1 for 4 h for the compositions detailed in Table 6.

TABLE 6

| Example | HBr | Ru₃(CO)₁₂ | PBu₄Br |
|---|---|---|---|
| No. | mg (mmol) | mg (mmol) | g (mmol) |
| 39 | 0.0 (0.00) | 250.1 (0.39) | 14.999 (44.2) |
| 40 | 9.4 (0.12) | 249.6 (0.39) | 14.961 (44.1) |
| 41 | 23.8 (0.29) | 250.0 (0.39) | 14.900 (43.9) |
| 42 | 47.5 (0.59) | 250.1 (0.39) | 14.805 (43.6) |
| 43 | 72.0 (0.89) | 250.4 (0.39) | 14.701 (43.3) |
| 44 | 83.5 (1.03) | 250.2 (0.39) | 14.652 (43.2) |
| 45 | 95.8 (1.18) | 249.6 (0.39) | 14.602 (43.0) |
| 46 | 119.5 (1.48) | 250.5 (0.39) | 14.502 (42.7) |

The amount of certain products produced (methanol, ethanol, 1-propanol, and ethylene glycol) is shown graphically in FIG. 9 as a function of the molar ratio of HBr/Ru.

### Examples 47-51

A series of five experiments was run to examine the effect of HI as a promoter on the hydrogenation of carbon monoxide with Ru₃(CO)₁₂ catalyst in molten [Bu₄P]I. A procedure similar to that described in Example 6 was carried out in each case with a reaction temperature of 195° C., CO/H₂ pressure of 3,626 psi (25 MPa), and CO/H₂ volumetric ratio of 1:1 for 4 h for the compositions detailed in Table 7.

TABLE 7

| | HI | Ru₃(CO)₁₂ | PBu₄I |
|---|---|---|---|
| Example No. | mg (mmol) | mg (mmol) | g (mmol) |
| 47 | 0.0 (0.00) | 249.7 (0.39) | 17.078 (44.2) |
| 48 | 37.4 (0.29) | 249.8 (0.39) | 16.965 (43.9) |
| 49 | 74.8 (0.58) | 250.1 (0.39) | 16.851 (43.6) |
| 50 | 112.7 (0.87) | 250.2 (0.39) | 16.738 (43.3) |
| 51 | 149.7 (1.17) | 250.0 (0.39) | 16.624 (43.0) |

The amount of certain products produced (methanol, ethanol, 1-propanol, and ethylene glycol) is shown graphically in FIG. 10 as a function of the molar ratio of HI/Ru.

The invention has been described in detail with particular reference to preferred embodiments thereof, but it will be understood that variations and modifications can be effected within the spirit and scope of the invention.

We claim:

1. A catalyst system for making one or more alkanols from a mixture of carbon monoxide and hydrogen, the catalyst system comprising:
a ruthenium compound and a halogen compound dispersed in a low-melting tetraorganophosphonium salt,
wherein the halogen compound is capable of generating HX under reaction conditions and is present in an amount effective to increase production of the one or more alkanols compared to a catalyst system without the halogen compound, and
wherein X is Cl, Br, or I.

2. The catalyst system according to claim 1, wherein the halogen compound is present in an amount sufficient to produce an HX to Ru atom molar ratio of 0.05:1 to 3:1 where X is Cl.

3. The catalyst system according to claim 1, wherein the halogen compound is present in an amount sufficient to produce an HX to Ru atom molar ratio of 0.1:1 to 2.5:1 where X is Cl.

4. The catalyst system according to claim 1, wherein the halogen compound is present in an amount sufficient to produce an HX to Ru atom molar ratio of 0.05:1 to 1:1 where X is Br or I.

5. The catalyst system according to claim 1, wherein the halogen compound is present in an amount sufficient to produce an HX to Ru atom molar ratio of 0.1:1 to 0.9:1 where X is Br or I.

6. The catalyst system according to claim 1, wherein the halogen compound is present in an amount sufficient to produce an HX to Ru atom molar ratio of 0.2:1 to 0.8:1 where X is Br or I.

7. The catalyst system according to claim 1, wherein the ruthenium compound is selected from the group consisting of anhydrous ruthenium(IV) dioxide, ruthenium(IV) dioxide hydrate, ruthenium(VIII) tetraoxide, ruthenium(III) trichloride hydrate, ruthenium acetate, ruthenium propionate, ruthenium(III) acetylacetonate, triruthenium dodecarbonyl, bis[ruthenium(tricarbonyl)dichloride], bis[ruthenium(tricarbonyl)dibromide], and bis[ruthenium(tricarbonyl)diiodide].

8. The catalyst system according to claim 1, wherein the halogen compound is hydrogen iodide or hydrogen bromide.

9. The catalyst system according to claim 1, wherein the halogen compound is a triorganophosphonium salt having the general formula (II):
[R₁R₂R₃PH][X²] (II)
wherein
R₁, R₂, and R₃ are each independently selected from C₁-C₂₄ alkyl or aryl hydrocarbon groups or functionalized alkyl or aryl groups containing ether, alcohol, ketone, carboxylic acid or ester, amine, amide, thioether, phosphine oxide, nitrile, heteroaromatic, or fluorocarbon groups; and
X² is chloride, bromide, or iodide.

10. The catalyst system according to claim 9, wherein X² is bromide.

11. The catalyst system according to claim 9, wherein the triorganophosphonium salt is tributylphosphonium bromide.

12. The catalyst system according to claim 9, wherein the molar ratio of the triorganophosphonium salt to Ru atom ranges from 0.2:1 to 0.6:1.

13. The catalyst system according to claim 1, wherein the tetraorganophosphonium salt has the general formula (I):
[R₁R₂R₃R₄P][X¹] (I)
wherein
R₁, R₂, R₃, and R₄ are each independently selected from C₁-C₂₄ alkyl or aryl hydrocarbon groups or functionalized alkyl or aryl groups containing ether, alcohol, ketone, carboxylic acid or ester, amine, amide, thioether, phosphine oxide, nitrile, heteroaromatic, or fluorocarbon groups; and
X¹ is chloride, bromide, or iodide.

14. The catalyst system according to claim 13, wherein X¹ is bromide.

15. The catalyst system according to claim 13, wherein the tetraorganophosphonium salt is tetrabutylphosphonium bromide.

16. The catalyst system according to claim 13, wherein the tetraorganophosphonium salt has a melting point of 180° C. or lower.

17. A process for preparing one or more alkanols, comprising:
contacting a mixture of carbon monoxide and hydrogen with a catalyst system comprising a ruthenium compound and a halogen compound dispersed in a low-melting tetraorganophosphonium salt under conditions effective to produce one or more alkanols,
wherein the halogen compound is capable of generating HX under reaction conditions and is present in an amount effective to increase production of the one or more alkanols compared to a catalyst system without the halogen compound, and
wherein X is Cl, Br, or I.

18. The process according to claim 17, wherein the conditions effective to produce one or more alkanols comprise a CO/H₂ pressure of at least 7 MPa and a temperature of at least 150° C.

19. The process according to claim 17, wherein the conditions effective to produce one or more alkanols comprise a CO/H₂ pressure of at least 14 MPa and a temperature of at least 180° C.

20. The process according to claim 17, wherein the halogen compound is present in an amount sufficient to produce an HX to Ru atom molar ratio of 0.1:1 to 2.5:1 where X is Cl.

21. The process according to claim 17, wherein the halogen compound is present in an amount sufficient to produce an HX to Ru atom molar ratio of 0.2:1 to 0.8:1 where X is Br or I.

22. The process according to claim 17, wherein the ruthenium compound is a ruthenium carbonyl.

23. The process according to claim 17, wherein the halogen compound is hydrogen iodide or hydrogen bromide.

24. The process according to claim 17, wherein the halogen compound is tributylphosphonium bromide.

25. The process according to claim 17, wherein the tetraorganophosphonium salt is tetrabutylphosphonium bromide.

26. The process according to claim 17, wherein the alkanol is methanol or ethanol.
